(19) United States
(12) Patent Application Publication
(10) Pub. No.: US 2008/0097411 A1
(43) Pub. Date: Apr. 24, 2008

(54) Catheter assemblies having sized sheaths

(76) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/526,056

(22) Filed: Sep. 25, 2006

(51) Int. Cl. A61M 27/00

(52) U.S. Cl. 604/544

(57) ABSTRACT

Devices and methods are disclosed for a catheterization process, particularly useful for self-performed catheterizations. A catheter is enclosed in a sheath made with a specific thickness. This sheath is thick enough to avoid rips and tears from normal use, yet thin enough to allow those with even limited manual dexterity to use the catheter with ease.

### BACKGROUND OF THE INVENTION

1. Field of the Invention

The present invention relates generally to catheter assemblies. More particularly, the present invention relates to catheter assemblies having sheaths with particular thickness.

2. Background of the Invention

It has become relatively commonplace for the occasional, intermittent or periodic catheterization of an individual's urinary bladder to be employed, as opposed to placement and maintenance of an indwelling catheter that continuously drains urine from the bladder. Short-term or repeated catheterization is appropriate, or even required, for many persons who are in a hospital setting, nursing home, doctor's office, rehabilitation facility, or in their own home. For example, a patient is sometimes catheterized in order to treat urinary retention, evacuate urine, or to obtain a sterile urine specimen from a patient.

The need for intermittent catheterization of an individual sometimes arises due to problems typically associated with long-term use of indwelling catheters such as infections, urethral damage, and bladder damage. Long-term use of an indwelling catheter is also a risk factor for bladder cancer. This is often the case for persons having a neurogenic urinary condition such as in a spinal cord injury, multiple sclerosis, stroke, trauma, or other brain injury. Other conditions that interfere with the individual's ability to voluntarily void the bladder may also arise post-surgically or as a result of benign prostatic hypertrophy or diabetes. Many of these affected individuals are capable of and would prefer to perform self-catheterization. For many, the level of risk and discomfort of repeated catheterizations carried out over the course of a day (at 3-6 hour intervals, for example) are offset by the accompanying convenience, privacy, and self-reliance achieved with self-catheterization. However, some major difficulties arise in the current self-catheterization techniques. These difficulties include the lack of satisfactory catheterization kits, the problem of maintaining the required level of sanitation during the procedure, difficulty in handling the catheter during insertion, and the difficulty of performing the procedure under conditions of restricted space and privacy.

In the assisted or non self-catheterizations presently employed in hospitals, use of a catheterization tray is common practice. This tray typically includes a sterile drape, gloves, a conventional catheter, antiseptic solution, swabs, lubricant, forceps, an underpad, and a urine collection container. Assisted catheterization is usually performed with the patient in a supine position. The nurse typically grabs the patient's genitalia with one hand while using the other hand to insert the catheter into the patient's urethra. Thus, the nurse has only limited dexterity for manipulating the catheter in and around the patient's urethra. Maintaining a sterile field using “sterile technique” during the procedure can be a problem. The “cath tray” procedure is impractical for use with some individuals having certain spastic and voluntary muscle disorders.

Many individuals with spinal cord injuries or other neurological diseases routinely perform intermittent catheterization several times a day using conventional catheters or kits and the “clean technique.” In the clean technique, the urethral area is initially swabbed with an antiseptic and efforts are made to avoid contamination of the catheter during the procedure. The user's hands are not sterile and a sterile field is not maintained. Clean technique is used instead of sterile technique, generally, for two reasons. First, it is very difficult, if not impossible, for individuals performing self-catheterization to adhere strictly to sterile technique. Secondly, these individuals are required to catheterize themselves between 3 and 6 times a day. The cost of a new sterile catheter and the accessories required to perform sterile catheterization can become excessively expensive for some users. Sometimes an individual will reuse a “cleaned” catheter. As a result, the use of “clean technique” often results in contamination and subsequent infection of the urinary tract, causing significant morbidity and cost to the patient and society.

Thus, there is a need in the healthcare industry for a catheter assembly that enables all users, particularly those users with limited dexterity, to grip the catheter assembly and insert a contamination free catheter with decreased difficulty. Further, the catheter assembly should be easy to use and control, while having minimal parts that interfere with the catheterization process.

One technique of maintaining complete sterility is the use of a sheath to cover the catheter during the catheterization process. The sheath allows the user to handle the catheter without making actual contact with it.

Such sheaths come in various textures and thicknesses. Regular use reveals that some sheaths which are vulnerable to rips and tears which compromise the integrity of the sheath. Maintaining complete sterility is one of the main purposes of the sheath, and any compromise defeats its purpose. Since the catheter is meant for use multiple times a day, any risk in tears from normal use is unacceptable.

Other conventional sheaths are difficult to pull back during insertion of the catheter. When inserting the catheter, the sheath is pulled back and bunches up near the distal end. Towards the end of this process the bunch of sheath can get large, making it difficult to pull the sheath back all the way. This challenge is magnified for individuals with limited manual dexterity, who represent a significant portion of the market for sheathed catheters.

### SUMMARY OF THE INVENTION

The present invention seeks to address some of the shortcomings of conventional catheters which have protective sheaths. One such way which is described herein is through presentation of a given range in which these problems are eliminated. A sheath of a thickness large enough to avoid rips and tears from normal use, yet thin enough to allow those with even limited manual dexterity to use the catheter with ease is utilized in a catheter system. The sheath will remain in-between the user's hand and the catheter during insertion preventing direct contact with the catheter. The sheath is also impervious to body fluids, atmospheric air, and microorganisms, which ensures sterility after manufacture. This invention can be utilized in all catheter assembly systems including but not limited to, indwelling and intermittent catheter systems, as well as in-patient and out-patient designs.

Furthermore, the present invention can include components that further ease the catheterization process. Particular exemplary components include lubrication so the catheter slides smoothly down the urinary tract, and a guiding tip, which may also have a lubricant reservoir, to give the user something solid to line up the catheter to the urethra. Also, a hydrophilic coating is used on the catheter of certain embodiments to hold the lubricant onto the catheter while in the urinary tract.

An exemplary embodiment of the present invention is a sheath of specified thickness with a textured surface. A textured surface is employed in the sheath that gives these users enough gripping ability to easily hold the sheath with their hand(s) while pulling the catheter through. This allows users with lower manual dexterity to complete the self-catheterization process efficiently, and with ease.

Another exemplary embodiment of the present invention is a gas-permeable sheath of a specified thickness. A catheter using a sheath made from a gas-permeable, yet liquid-impermeable, material will allow excess air inside the catheter to leak through the sheath without compromising the integrity, and thus the sterility, of the sheath, relieving the build-up of air at the distal end of the sheath.

### BRIEF DESCRIPTION OF THE DRAWINGS

FIG. 1A shows an external perspective view of a catheter surrounded by a sheath according to an exemplary embodiment of a conventional assembly.

FIG. 1B shows an external perspective view of a catheter surrounded by a sheath with the sheath pulled back to expose the catheter.

FIGS. 2A-2G show close-up views of a surface texture of a sheath according to various exemplary embodiments of the present invention.

FIG. 2H shows an external perspective view of a catheter surrounded by a sheath with a box showing the section exploded in FIGS. 2A-2G.

FIG. 3A shows an external perspective view of a catheter surrounded by a gas-permeable sheath according to an exemplary embodiment of the present invention.

FIG. 3B shows an external perspective view of a catheter surrounded by a gas-permeable sheath with the sheath pulled back to expose the catheter.

FIG. 4A shows an external perspective view of a catheter surrounded by a sheath attached to a guide tip at the proximal end according to an exemplary embodiment of the present invention.

FIG. 4B shows a close-up view of a guiding tip according to an exemplary embodiment of the present invention.

### DETAILED DESCRIPTION OF THE INVENTION

The present invention is a sheath 100 for a catheter 110 or catheter assembly of a specified thickness. In particular, the present invention includes devices and methods for urinary catheterization for patients who want to self-catheterize in a sterile and safe manner, yet do not have the manual dexterity to complete the process efficiently. In order to achieve the level of sterility required to avoid infection, a sheath 100 is used to cover the portion 180 of the catheter that is insertable into the urethra. The sheath 100 can cover the catheter 110 from time of storage until full insertion. The user can pull the catheter 110 through the sheath 100 without touching the catheter 110 itself.

The thickness of the sheath according to the present invention yields a sheath 100 that is strong enough to withstand the forces of normal use, and with enough flexibility to allow even those with limited manual dexterity to slide the sheath 100 to the distal end of the catheter 102 during insertion. To avoid rips and tears in the sheath 100 from normal use, a sheath 100 thickness of 0.0020″ may be used as a minimum sheath thickness. Avoiding problems of pulling the sheath 100 back to expose the catheter 110 depends on the user's manual dexterity. A wide range of thicknesses is possible, but users with limited manual dexterity are usually not comfortable with a sheath thickness greater than 0.0065″. Further experiments have shown that a sheath thickness of 0.0040″ to 0.0045″ allows those with limited manual dexterity ease of use while still offering a strong resistance against rips and tears.

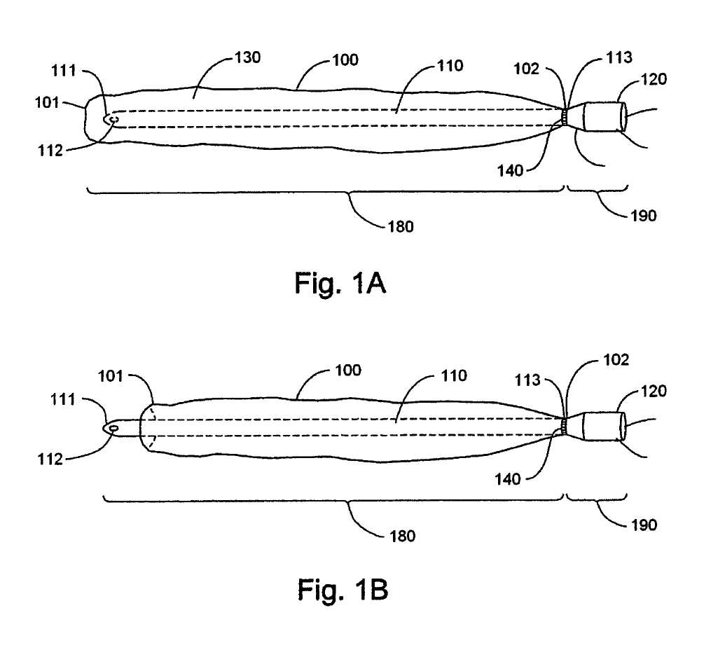

An exemplary embodiment of a conventional assembly for a catheter with a sheath is shown in FIG. 1A. The proximal end of the sheath 101 surrounds the proximal tip of the catheter 111 and is dosed at the end. The distal end of the sheath 102 is attached near the distal end of the urethra insertable portion of the catheter 180 with plastic or elastomeric ties or bands 140 or heat sealed. The distal end of the sheath 102 could also be attached to the outlet 120 if the catheter employs one. This outlet 120 could then be used to attach a urine bag or the like.

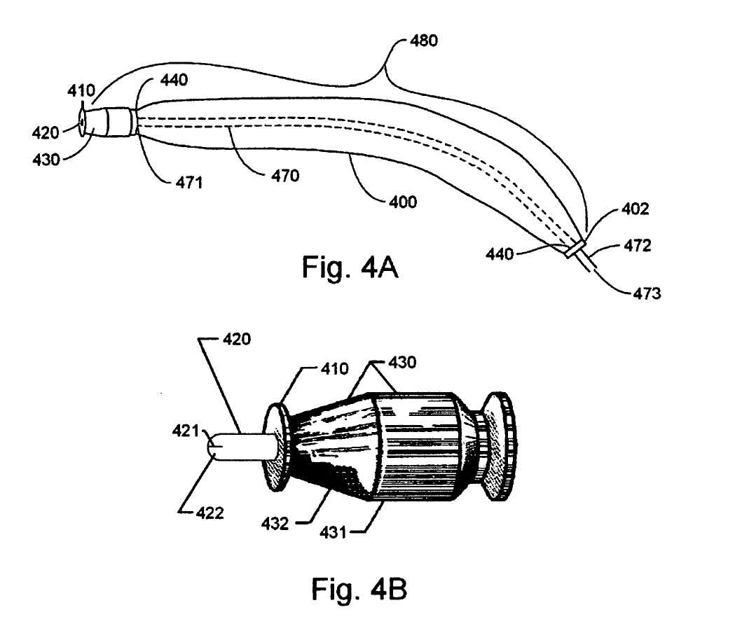

In order to perform a catheterization using this device the user should first open the proximal end of the sheath 100, exposing the proximal end of the catheter 111, as shown in FIG. 1B. The user then holds the proximal tip of the catheter 111 with the sheath 100 between the user's hand and the catheter 110 and pulls the sheath 100 with the other hand. As the user pulls the sheath 100, which is attached to the catheter at its distal end 113, the catheter 110 will be pushed through the sheath 100 and into the urethra, causing the sheath 100 to bunch up at the proximal end 101. For embodiments utilizing a guiding tip FIG. 4 at the proximal end of the catheter 111, a catheterization can be performed without opening the proximal end of the sheath 101. The user can simply push the catheter 110 through the guiding tip 150 and into the urethra.

This process continues until the catheter 110 runs all the way through the urethra and into the bladder. Once inside, fluid from the bladder will stream into the catheter through the opening 112 at the proximal tip of the catheter 111. Provided the catheter distal end 113 is lower in altitude than the catheter proximal end 111, fluid will flow through the catheter, out the outlet 120, and into a receptacle. When the bladder has been drained of all fluid the catheter 110 is then pulled out of the urethra by the user, and disposed.

Another exemplary embodiment of the present invention is a textured sheath of specified thickness, such as in simultaneously pending U.S. patent application Ser. No. 10/______, entitled “Catheter Assemblies Having Protective Sheaths”, by the same inventor as in the present invention, and which is incorporated by reference herein in its entirety. This texture is essentially any topography of a non-uniform elevation. Uniform elevation would be any surface that is flat and smooth, which increases the likelihood that the surface is either slippery or can be made slippery quickly by contact with moisture, either on the user's hand or elsewhere. The textured sheath seeks to employ surfaces that have ridges, bumps, notches or any other type of substantially non-flat or non-smooth surface to enhance the contact and grip with a user's hand and fingers.

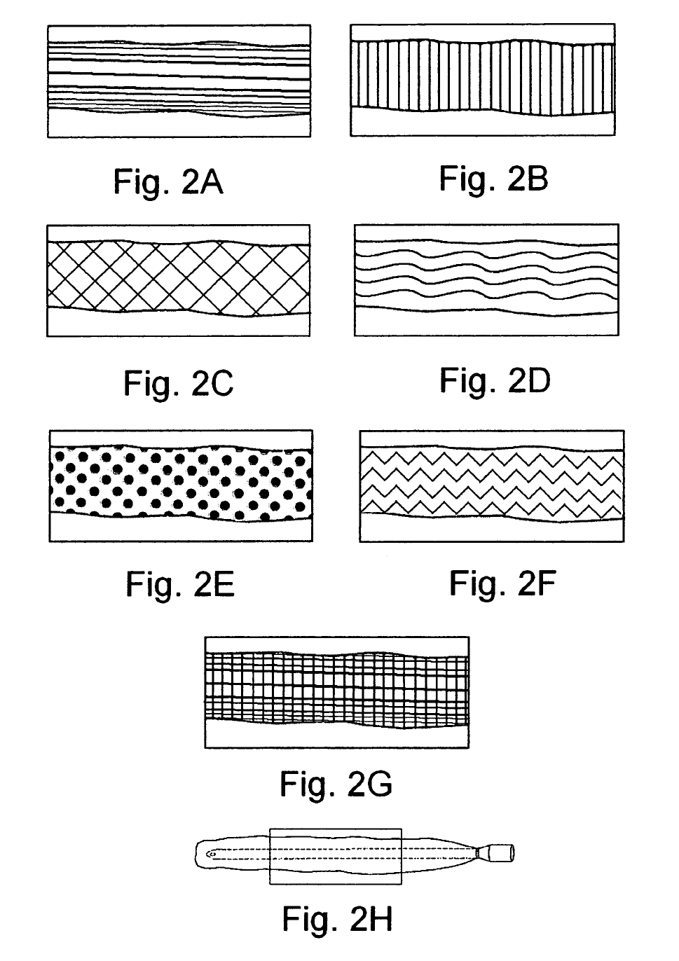

There are many possible textures for the sheath 100. These textures can be placed on the inside of the sheath as well, providing a user with grip on the catheter. FIG. 2A shows a series of ribs running parallel with the surface of the sheath 100, which is the basic technique for providing grip, preventing slipping perpendicular to the movement of the catheter 110. If used on the inside of the catheter, the parallel ribs will give the user enough grip to manipulate the catheter while still allowing it to slide forward through the sheath. FIG. 2B shows a series of ribs running perpendicular to the surface of the sheath 100. The perpendicular ridges prevent slipping against the movement of the catheter 110. It also allows the sheath to fold up more easily as the sheath compresses towards the proximal end during insertion. FIG. 2C shows a series of ribs interwoven in a hatch pattern, which prevents slipping in any direction. FIG. 2G shows another embodiment of a hatch pattern. FIG. 2D shows a series of wavy ribs running longitudinal with the surface of the sheath 100, which performs the functions of the pattern in FIG. 2A, but with an added ability to prevent movement in the transverse direction. FIG. 2E shows a series of bumps or projections covering the surface of the sheath 100, which provides the user with larger obtrusions for even more grip. If the surface of the sheath is wet, these bumps will serve as dry pads for the fingers while the moisture rests in the valleys in between. FIG. 2F shows a series of ribs in a tread-like pattern, much like the bottom of a pair of running shoes or a tire, which is another technique of preventing slipping in any direction. Many of these patterns have ribs or ridges or the like. These ribs or ridges should stand high enough to prevent slipping it the given direction, but not so high as to interfere with the user's ability to move the catheter through the center or the sheath's ability to collapse to the proximal end during insertion.

Yet another exemplary embodiment of the present invention is a sheath of specified thickness made from a gas-permeable material, such as in simultaneously pending U.S. patent application Ser. No. 10/______, entitled “Catheter Assembly”, by the same inventor as in the present invention, and which is incorporated by reference herein in its entirety. One exemplary group of materials with this gas-permeability property includes silicon-based organic polymers, also known as silicon oils. They are flexible, strong, and can retain their strength through a wide range of temperatures. In addition to gas permeability, they are also very resistant to chemicals and ultraviolet rays. Silicon-based organic polymers are liquid-impermeable and do not allow bacteria or other harmful substances to pass, making them useful in medical applications such as the present invention.

One such silicon-based organic polymer for use in the present invention is polydimethylsiloxane, also known as dimethicone or its trade name, SILICON ELASTOMER. Its density ranges from 1.1 to 1.5 g/cm³. Since the density of polidimethylsiloxane is proportional to its gas-permeability, a less dense version, 1.1 to 1.3 g/cm³ is preferred.

Another group of materials suitable for this invention is microporous polyolefins. Unlike regular polyolefins, these microporous polyolefins have tortuous sub micron-size passageways extending from one surface side to the other. This allows the passage of gas and vapor while prohibiting the passage of particles and liquids. The microporous polyolefin material can be made by taking a microporous polyolefin matrix and sufficiently filling the pores with a moisture-vapor permeable, liquid-impermeable, hydrophilic material to prevent the passage of water and other liquids through the polyolefin material while readily permitting moisture vapor as in U.S. Pat. No. 4,613,544, entitled “Waterproof, moisture-vapor permeable sheet material and method of making the same”, issued to Burleigh, which is incorporated by reference herein in its entirety.

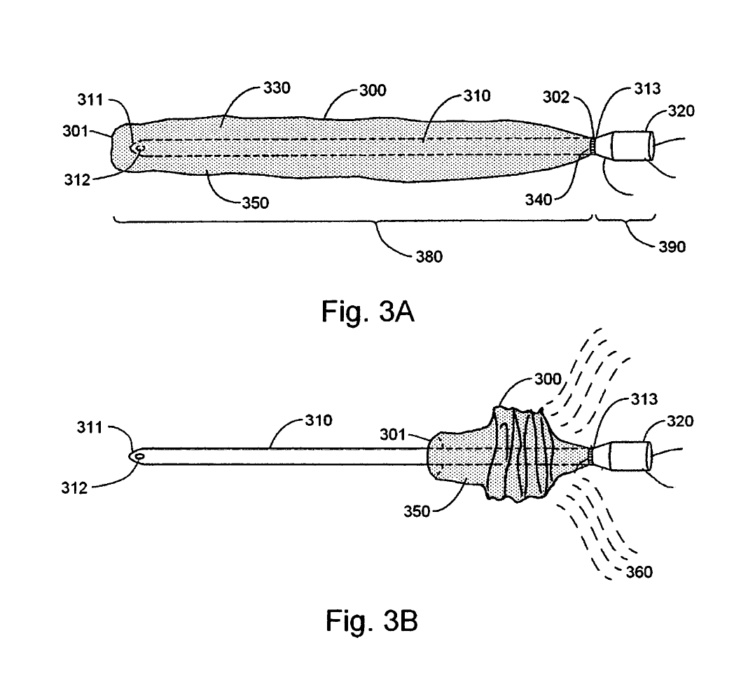

FIG. 3A features a sheath 300 made from a gas-permeable, liquid-impermeable material 350. This material 350 will allow the air built-up inside the sheath to escape to the outside atmosphere 360 at a rate fast enough for the user to complete the catheterization process without undue pause, as illustrated in FIG. 3B.

In yet another exemplary embodiment, the sheath 100 will be filled with enough lubricant 130 to coat the insertable length of the catheter 180. This will be a water-based lubricant of the type used on rectal thermometers and enemas such as KY-JELLY®. As the catheter 110 is pushed through the sheath 100 the lubricant 130 is pushed through as well, lubricating the insertable portion of the catheter 180 on its way into the urethra. The lubricant 130 will ease the process of sliding the catheter 110 into the urethra by reducing the friction between the catheter 110 and the urethra. By reducing the friction the user can insert the catheter 110 faster and with less pain.

In yet another exemplary embodiment, featured in FIG. 4A, the catheter 470 will have a guiding tip 430 at the proximal end of the catheter 471. The guiding tip 430 helps the user hold the catheter 470 in place while inserting it into the urethra. The guiding tip 430 has a throughbore 420 in the center which the catheter 470 can slide through.

One such embodiment of the guiding tip 430 is illustrated in FIG. 4B. At the proximal end of the guiding tip is a collar 410, with a size of about ten to fifteen millimeters, which rests on the outside of the urethra during insertion. At the proximal end of the collar 410 is a short tube 420 just wider than the catheter 470. This tube 420 ends in a rounded top with two cuts in the top 421. When the catheter 470 is pushed through the top the tube 420 splits into four tabs 422, allowing the catheter 470 to pass. Towards the distal end of the guiding tip 430 there is also a reservoir portion 430. The distal half of the reservoir is a hollow cylinder 431 while the proximal half is a hollow frustoconical section 432. The reservoir portion 431 contains the same lubricant 130 held inside the sheath 400. This makes the guiding tip 430 longer and bulkier and gives the user more to hold onto while sliding the catheter 470 through. On the outside of the reservoir 431, texture can be added for extra gripping. Other embodiments of the guiding tip can be found in U.S. Pat. No. 6,090,075, entitled “Disposable urinary catheterization assembly”, issued to House, which is incorporated by reference herein in its entirety.

In other embodiments the catheter 110 will be coated with a hydrophilic substance, commonly known as hydrogel, particularly useful on indwelling catheters. This hydrophilic coating helps the catheter 110 to hold the lubricant 130 on its surface while inside the urinary tract. One such hydrophilic substance that can be used is agarose, known also under its trade name BIOGEL A.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A catheter assembly comprising:
a catheter; and
a sheath, having a thickness between 0.0020″ and 0.0065″, surrounding and enclosing at least a portion of the catheter.

2. The catheter assembly in claim 1, wherein the sheath has a thickness between 0.0040″ and 0.0045″.

3. The catheter assembly in claim 1, wherein the sheath has a textured surface.

4. The catheter assembly in claim 3, wherein the texture is a non-smooth surface.

5. The catheter assembly in claim 4, wherein the texture is composed of ridges.

6. The catheter assembly in claim 1, wherein the sheath is made of a liquid-impermeable material that is sterile or able to withstand sterility treatment, impermeable to bacteria and other contaminants, and gas-permeable.

7. The catheter assembly in claim 1, wherein the sheath is made from a silicon-based organic polymer.

8. The catheter assembly in claim 7, wherein the silicon-based organic polymer is polymethylsiloxane.

9. The catheter assembly in claim 8, wherein the polymethylsiloxane has a density of 1.1 to 1.3 g/cm³.

10. The catheter assembly in claim 1, wherein the sheath is made from a microporous polyolefin.

11. The catheter assembly in claim 10, wherein the polyolefin is polypropylene.

12. The catheter assembly in claim 11, wherein the polypropylene density is 0.87 to 0.9 g/cm³.

13. A urinary catheter assembly comprising:
a sterile, flexible catheter; and
a sheath, having a thickness between 0.0020″ and 0.0065″, surrounding and enclosing at least a portion of the catheter.

14. The catheter assembly in claim 13, wherein the sheath has a thickness between 0.0040″ and 0.0045″.

15. The catheter assembly in claim 13, wherein the sheath has a textured surface.

16. The catheter assembly in claim 13, wherein the sheath is made of a liquid-impermeable material that is sterile or able to withstand sterility treatment, impermeable to bacteria and other contaminants, and gas-permeable.

17. The catheter assembly in claim 13, wherein the sheath is made from a silicon-based organic polymer.

18. The catheter assembly in claim 13, wherein the sheath is made from a microporous polyolefin.

19. An intermittent urinary catheter assembly comprising:
a sterile, flexible catheter; and
a sheath, having a thickness between 0.0020″ and 0.0065″, surrounding and enclosing at least an insertable portion of the catheter.

20. The catheter assembly in claim 19, wherein the sheath has a thickness between 0.0040″ and 0.0045″.
